(19) United States
(12) Patent Application Publication
(10) Pub. No.: US 2021/0361715 A1
(43) Pub. Date: Nov. 25, 2021

(54) USE OF EXOSOME DERIVED FROM MESENCHYMAL STEM CELLS CO-CULTURED WITH MELATONIN IN PREVENTION AND TREATMENT OF CHRONIC KIDNEY DISEASE

(71) Applicants: Sang Hun LEE, Yongin-si (KR); STEM BIO CO., LTD., Asan-si (KR)

(72) Inventor: Sang Hun Lee, Yongin-si (KR)

(73) Assignees: STEM BIO CO., LTD., Asan-si (KR); Sang Hun LEE, Yongin-si (KR)

(21) Appl. No.: 16/906,839

(22) Filed: Jun. 19, 2020

(30) Foreign Application Priority Data
May 21, 2020 (KR) 10-2020-0060775

(51) Int. Cl. A61K 35/28; C12N 5/0775; A61P 13/12

(52) U.S. Cl. CPC A61K 35/28; C12N 5/0665; A61P 13/12; C12N 5/0668; C12N 5/0663

(57) ABSTRACT

A new use of exosomes extracted from mesenchymal stem cells derived from a healthy individual co-cultured with melatonin or a culture solution thereof, for the treatment of chronic kidney disease is disclosed. A pharmaceutical composition containing the exosomes extracted from mesenchymal stem cells derived from a healthy individual co-cultured with melatonin or a culture solution thereof as an active ingredient and a method for preparing the pharmaceutical composition are disclosed. The exosomes promote the proliferation of mesenchymal stem cells derived from a chronic kidney disease patient or increase the survival rate of the patient.

### CROSS REFERENCE TO RELATED APPLICATION

This application claims priority based on Korean Patent Application No. 10-2020-0060775 filed May 21, 2020.

### BACKGROUND OF THE DISCLOSURE

### Field of the Disclosure

The present invention relates to a use of exosomes extracted from mesenchymal stem cells derived from a healthy individual co-cultured with melatonin or a culture solution thereof, for the treatment of chronic kidney disease. More specifically, the present invention relates to a pharmaceutical composition for treatment or prevention of chronic kidney disease comprising exosomes extracted from mesenchymal stem cells derived from a healthy individual co-cultured with melatonin or a culture solution thereof as an active ingredient, a method for preparing the pharmaceutical composition for treatment or prevention of chronic kidney disease, and a method for promoting the proliferation of mesenchymal stem cells derived from a chronic kidney disease patient or increasing the survival rate of the patient.

### Related Art

A stem cell refers to a type of cell, which, while remaining undifferentiated into specific cells, has the potential to differentiate into all kinds of cells that make up the body, such as nerves, blood, cartilage, etc., as necessary. There are largely two methods to obtain these stem cells. The first is to obtain from an embryo generated from a fertilized egg, and the second is to recover the stem cells that are retained in each part of our adult body. Although there are differences in terms of function, all stem cells can be differentiated into different types of cells. However, the adult stem cells extracted from adults have advantages in that they are relatively free from ethical problems and can be cultured in large amounts.

Melatonin is an endogenous indoleamine hormone secreted by the pineal gland and it is secreted by various tissues, such as bone marrow, liver, intestines, placenta, ovaries, and testes. In particular, it is known that melatonin easily crosses all physiological barriers, including cell membranes and cerebral vessels, and enters the cerebrospinal fluid of the third ventricle through the pineal gland as well as being released into the bloodstream. Melatonin has been identified to regulate several physiological functions including sleep, circadian rhythm, immune defenses, and neuroendocrine actions. In addition, studies have shown that melatonin has the effects of antioxidation, anticancer, anti-inflammation, and regulation of autophagy. In particular, studies have shown that melatonin improves the efficacy of stem cell-based treatment in diseases, such as myocardial infarction and acute lung ischemia. However, the pathophysiological mechanisms by which melatonin enhances the biological activity of stem cells are still not clear.

Normal prion protein (cellular prion protein; PrPC) is found in an image to be on a cell membrane through the glycolipid on the cell surface or embedded in the cell membrane. Abnormally altered prion proteins (PrPSc) are associated with the development of prion diseases and neurodegenerative diseases, and recently, research reports have shown that cellular prion proteins serve as a major factor that plays a fundamental role in stem cell proliferation and self-regeneration and that they play a protective role against neurodegeneration. In particular, studies have shown that prion proteins are involved in differentiation of stem cells and progenitor cells, neurogenesis, and angiogenesis.

The exosomes or extracellular vesicles, in which various bioactive factors that control the behavior of cells are contained, include intercellular signaling functions, and research on their components and functions is actively underway. Cells release various membrane-type vesicles into the extracellular environment, and these released vesicles are commonly called extracellular vesicles. The extracellular vesicles are cell membrane-derived vesicles, ectosomes, shedding vesicles, microparticles, exosomes, etc., and in some cases, they are distinguished from exosomes. Exosomes are vesicles of several tens to several hundreds of nanometers in size consisting of a phospholipid bilayer having the same structure as the cell membrane, and they include therein proteins, mRNAs, miRNAs, etc., called exosome cargo. The exosome cargo includes a wide range of signaling elements, and these signaling elements are known to be cell type specific and regulated differently according to the environment of their secretory cells. Exosomes are intercellular signaling media secreted by cells, and the various cellular signals transmitted through the exosomes are known to regulate cell behaviors including target cell activation, growth, migration, differentiation, dedifferentiation, apoptosis, and necrosis. Exosomes include specific genetic materials and bioactive factors depending on the nature and conditions of the cells from which they are derived. In the case of the exosomes derived from proliferating stem cells, they can regulate cell behaviors such as cell migration, proliferation, and differentiation, and the characteristics of stem cells associated with tissue regeneration are reflected therein.

### Prior Art

1. Patent Document (KR 10-2017-0110579 A)

### SUMMARY OF THE DISCLOSURE

As such, the present inventors have made great efforts to develop effective treatment methods for chronic kidney disease, and as a result, they have found that exosomes extracted from mesenchymal stem cells derived from a healthy individual co-cultured with melatonin can increase the expression of proteins associated with angiogenesis, anti-inflammation, and cell invasion as well as recovering mitochondrial functions, cellular senescence, and cell proliferative potential, thereby completing the present invention.

An object of the present invention is to provide a pharmaceutical composition for treatment or prevention of chronic kidney disease, including exosomes extracted from mesenchymal stem cells derived from a healthy individual co-cultured with melatonin or a culture solution thereof as an active ingredient.

Additionally, an object of the present invention is to provide a method for preparing the pharmaceutical composition for treatment or prevention of chronic kidney disease.

Additionally, an object of the present invention is to provide a method for promoting the proliferation of mesenchymal stem cells derived from a chronic kidney disease patient or increasing the survival rate of the patient.

As used herein, the term “mesenchymal stem cell” refers to a cell which has a potential of self-replication and a potential to differentiate into two or more types, and it can be classified into a totipotent stem cell, a pluripotent stem cell, and a multipotent stem cell. The mesenchymal stem cells from a healthy individual used in the present invention are heterologous mesenchymal stem cells extracted from allogeneic strains and cultured, and may be mesenchymal stem cells derived from bone marrow, adipose and muscle tissues, etc. and cultured in vitro.

As used herein, the term “aid for stem cell treatment” refers to a preparation that can be used supplementarily to enhance the effectiveness of stem cell therapeutics commonly used in the art. As used herein, the term “stem cell treatment” refers to a pharmaceutical drug used for the purpose of diagnosis, treatment, or prevention through the acts of changing the biological characteristics of cells, etc. using other methods (e.g., proliferation, culturing, and screening of living autologous, allogeneic, and heterogeneous cells in vitro) so as to restore the functions of cells and tissues, and the term “stem cell therapeutic” refers to a cell therapeutic in which embryonic stem cells or adult stem cells are used as a material for the cell therapeutic.

As used herein, the term “exosome” refers to a small (approximately 30-100 nm in diameter) vesicle of a membrane structure secreted from various cells, and refers to a vesicle that is released into the extracellular environment by the occurrence of a fusion between a polycyst and a plasma membrane. The exosome includes those which are naturally secreted or artificially secreted.

### BRIEF DESCRIPTION OF THE DRAWINGS

FIG. 1 is a graph showing the measurement results of the estimated glomerular filtration rate (eGFR) of healthy individuals and chronic kidney disease (CKD) patients.

FIGS. 2A to 2F show effect of chronic kidney disease (CKD) on the biological functions in mesenchymal stem/stromal cells (MSCs). FIG. 2A shows a result of senescence-associated β-galactosidase (SA-β-gal) staining in MSCs derived from healthy individuals (healthy MSCs) and patients with CKD (CKD-MSCs) (n=5). Scale bar=100 μm. Cellular senescence was determined by the number of SA-β-gal-positive cells. The values represent mean±standard error of the mean (SEM), **P<0.01. FIG. 2B shows the expression of p16, p21, and SMP30 in healthy MSCs and CKD-MSCs (n=3). The protein levels were quantified by densitometry and normalized to β-actin levels. The values represent mean±SEM, *P<0.05, **P<0.01. FIG. 2C shows a result of single-cell expansion assay in healthy and CKD-MSCs (n=5). Scale bar=100 μm. Proliferative potential was determined by the number of cells that migrated toward the periphery of the culture dish. The values represent mean±SEM, **P<0.01. FIG. 2D shows a result of BrdU incorporation in healthy and CKD-MSCs (n=5). The values represent the means±SEM. *P<0.05. FIG. 2E shows a result of s-phase flow cytometry (n=5) analysis for healthy and CKD-MSCs. The values represent mean±SEM, **P<0.01. FIG. 2F shows levels of p-Akt, p-ERK, p-FAK, and p-Src in healthy and CKD-MSCs (n=3). The proteins were quantified by densitometry normalized to total Akt, ERK, FAK, and Src levels, respectively. The values represent mean±SEM, *P<0.05, **P<0.01.

FIGS. 3A to 3D show characterization of exosomes isolated from healthy MSCs. FIG. 3A shows the expression of CD81 and CD63 in exosomes isolated from healthy MSCs (Con exosomes) and melatonin-treated healthy MSCs (MT exosomes). FIG. 3B shows a result of flow cytometry analysis for CD81 and CD63 in Con exosomes and MT exosomes (n=3). FIG. 3C and FIG. 3D shows Size distribution and Polydispersity index (P.I.) analysis of using dynamic light scattering. To determine the size distribution, exosomes were subjected to dynamic light scattering measurements using ElSZ-1000 (otsuka electronics, Kobe, Japan) (n=3).

FIGS. 4A-4H show melatonin increases the level of cellular prion protein (PrP^{C}) in MSC-derived exosomes via upregulation of miR-4516. FIG. 4A shows concentration of PrPc in serum of healthy individuals (n=40) and patients with CKD (stage 3; n=37) as measured by ELISA. The values represent mean±SEM, **P<0.01. FIG. 4B shows the level of PrPc in cell lysates and exosomes from healthy and CKD-MSCs (n=3) as measured by ELISA. The values represent mean±SEM, **P<0.01 compared with healthy MSCs; ##P<0.01 compared with healthy MSCs treated with melatonin; $$P<0.01 compared with untreated CKD-MSCs. FIG. 4C shows levels of PrPc in CKD-MSCs after treatment with melatonin, exosomes derived from healthy MSCs (Con exosomes), and exosomes derived from melatonin-treated healthy MSCs (MT exosomes) (n=3) as measured by ELISA. The values represent mean±SEM, **P<0.01 compared with PBS; ##P<0.01 compared with melatonin-treated; $$P<0.01 compared with Con exosomes. FIG. 4D shows a result of hierarchical clustering showing differential miRNA expression in Con exosomes and MT exosomes. FIG. 4E shows a result of the expression of miR-4516 in Con exosomes and MT exosomes (n=3) as measured by real-time PCR. The values represent mean±SEM, **P<0.01. FIG. 4F shows miR-4516 levels in healthy MSCs and CKD-MSCs after treatment with melatonin (n=3) as measured by real-time PCR. The values represent mean±SEM, **P<0.01 compared with untreated healthy MSCs; ##P<0.01 vs untreated CKD-MSCs; $$P<0.01 compared with melatonin-treated healthy MSCs. FIG. 4G and FIG. 4H show levels of PrPC in MT exosomes with/without miR-4516 inhibitor (n=5) as measured by ELISA. The values represent mean±SEM, **P<0.01 compared with Con exosomes; ##P<0.01 compared with MT exosomes. H, PrPc expression in CKD-MSCs after treatment with Con exosomes and MT exosomes (n=5) as measured by ELISA. The values represent mean±SEM, **P<0.01 compared with PBS; ##P<0.01 compared with Con exosomes; $$P<0.01 compared with MT exosomes.

FIGS. 5A-5D show expression of PrPc in healthy MSCs, CKD-MSCs, and exosomes derived from healthy MSCs via expression of miR-4516. FIG. 5A shows levels of miR-4516 in healthy MSCs after inhibition and overexpression of miR-4516 (n=3). The values represent mean±SEM, **p<0.01 compared to untreated healthy MSCs (negative control; N.C); ^{##}p<0.01 compared to healthy MSCs treated with miR-4516 inhibitor. FIG. 5B shows a result of expression of PrPc in healthy MSCs derived from healthy MSCs after inhibition and overexpression of miR-4516 (n=3). The values represent mean±SEM, **p<0.01 compared to untreated healthy MSCs (negative control; N.C); ^{##}p<0.01 compared to healthy MSCs treated with melatonin, ^{$$}p<0.01 compared to healthy MSCs treated with miR-4516 inhibitor. FIG. 5C shows a result of expression of PrP^{C} in healthy CKD-MSCs derived from healthy MSCs after inhibition and overexpression of miR-4516 (n=3). The values represent mean±SEM, **p^{##}p^{$$}p. FIG. 5D shows a result of expression of PrPc in healthy exosomes derived from healthy MSCs after inhibition and overexpression of miR-4516 (n=3). The values represent mean±SEM, **p^{##}p^{$$}p.

FIGS. 6A to 6F show that miR-4516 regulates the expression of PrPc through GP78-ubiquitination axis. FIG. 6A shows levels of GP78 in healthy MSCs after inhibition or overexpression of miR-4516. FIG. 6B shows level of GP78 in healthy MSCs after inhibition or overexpression of miR-4516 (n=3). The protein was quantified by densitometry normalized to β-actin level. The values represent mean±SEM, *p<0.05, **p<0.01 compared to untreated healthy MSCs (negative control; N.C); ^{##}p<0.01 compared to healthy MSCs treated with miR-4516 inhibitor. FIG. 6C shows co-immunoprecipitation analysis of PrPc bound to ubiquitin in healthy MSCs after inhibition or overexpression of miR-4516 (n=3). FIG. 6D shows a result of protein quantification by densitometry normalized to β-actin levels. The values represent mean±SEM, *p<0.05, **p<0.01 compared to untreated healthy MSCs (negative control; N.C); ^{##}p<0.01 compared to healthy MSCs treated with miR-4516 inhibitor. FIG. 6E shows expression of GP78 and PrPc in CKD-MSCs treated with PBS, CKD-MSCs treated with Con exosomes, CKD-MSCs treated with MT exosomes, and CKD-MSCs treated with MT exosomes+miR-4516 inhibitor (n=3). FIG. 6F shows a result of protein quantification by densitometry normalized to β-actin levels. The values represent mean±SEM, **p<0.01 compared to PBS; ^{#}p<0.05, ^{##}p<0.01 compared to Con exosomes; ^{$}p<0.05, ^{$$}p<0.01 compared to MT exosomes.

FIGS. 7A to 7F show effect of MT exosomes on mitochondrial function in CKD-MSCs via expression of PrPC. FIG. 7A shows a result of representative TEM images of mitochondria in CKD-MSCs after treatment with exosomes. Scale bar=1 μm. FIG. 7B shows percentage of abnormal mitochondria obtained from a TEM image (n=3). The values represent mean±SEM, **P<0.01 compared with PBS; ##P<0.01 compared with Con exosomes; $$P<0.01 compared with MT exosomes treated with siRNA against PRNP (MT exosomes+siPRNP). FIGS. 7C to 7F show activities of mitochondrial complex I (C) and IV (D) and SOD2 (E) in exosome-treated CKD-MSCs (n=3). The values represent mean±SEM, *P<0.05, **P<0.01 compared with PBS; #P<0.05, ##P<0.01 compared with Con exosomes; $$P<0.01 compared with MT exosomes+siPRNP. F, MitoSOX-positive cells quantified by flow cytometry in exosome-treated CKD-MSCs (n=5). The values represent mean±SEM, *P<0.05, **P<0.01 compared with PBS; ##P<0.01 compared with Con exosomes; $$P<0.01 compared with MT exosomes+siPRNP.

FIGS. 8A to 8D show MT exosomes protect cellular senescence in CKD-MSCs by upregulating PrP^{C}. FIG. 8A shows a result of SA-β-gal staining in CKD-MSCs treated with exosomes. FIG. 8B shows a result of cellular senescence was determined by the number of SA-β-gal-positive cells (n=5). Scale bar=100 μm. The values represent mean±standard error of the mean (SEM), **P<0.01 compared with PBS; ##P<0.01 compared with Con exosomes; $P<0.05 compared with MT exosomes+siPRNP. FIG. 8C shows a result of the expression of p16, p21, and SMP30 in CKD-MSCs treated with exosomes. FIG. 8D shows protein levels were quantified by densitometry normalized to β-actin levels (n=3). The values represent mean±SEM, **P<0.01 compared with PBS; ##P<0.01 compared with Con exosomes; $P<0.05, $$P<0.01 compared with MT exosomes+siPRNP.

FIGS. 9A to 9D show effects of MT exosomes on proliferation and cellular signaling in CKD-MSCs. FIG. 9A shows a result of single-cell expansion assay in CKD-MSCs treated with exosomes. Scale bar=100 μm. FIG. 9B shows the number of expanded cells in CKD-MSCs treated with exosomes (n=5). The values represent mean±SEM. **P<0.01 compared with PBS; ##P<0.01 compared with Con exosomes; $$P<0.01 compared with MT exosomes+siPRNP. FIG. 9C shows levels of p-Akt, p-ERK, p-FAK, and p-Src in CKD-MSCs treated with exosomes. FIG. 9D shows a result of the protein quantification by densitometry normalized to total Akt, ERK, FAK, and Src levels, respectively. The values represent mean±SEM, *P<0.05, **P<0.01 compared with PBS; #P<0.05, ##P<0.01 compared with Con exosomes; $P<0.05, $$P<0.01 compared with MT exosomes+siPRNP.

FIG. 10A is a table representing each target antibody of the angiogenesis antibody array-membrane test, which confirms the expression level of angiogenesis-associated proteins, in the autologous mesenchymal stem cells derived from a chronic kidney disease (CKD) patient pretreated with a pharmaceutical composition including the above exosomes, in which the parts with a change are indicated in color. FIG. 10B shows a result of dot-blot analysis of angiogenesis-mediated proteins in CKD-MSCs treated with PBS, Con exosomes, and MT exosomes. FIG. 10C shows the levels of vascular endothelial growth factor receptor 2 (VEGFR2; 2nd row, 7-8 line; blue box), VEGFR3 (3rd row, 7-8 line; blue box), interleukin 1 alpha (IL-la; 5th row, 3-4 line; yellow box), interferon-inducible T-cell alpha chemoattractant (I-TAC; 1st row, 5-6 line; yellow box), monocyte-chemotactic protein 3 (MCP-3; 2nd row, 5-6 line; yellow box), MCP-4 (3rd row, 5-6 line; yellow box), matrix metalloproteinase-1 (MMP-1; 4th row, 5-6 line; red box), MMP-9 (5th row, 5-6 line; red box), and urokinase receptor (uPAR; 1st row, 7-8 line; red box) in CKD-MSCs treated with PBS, Con exosomes, and MT exosomes (n=6). The values represent mean±SEM, **P<0.01 compared with PBS; ##P<0.01 compared with Con exosomes.

FIGS. 11A to 11H show MT exosome-treated CKD-MSCs increase the functional recovery in a murine hindlimb ischemia model with CKD. FIGS. 11A and 11B show TUNEL staining (A) and immunofluorescence staining for PCNA (B) using day 3 ischemic limb tissues (n=5). Scale bar=50 The values represent mean±SEM, *P<0.05, **P<0.01 compared with PBS; #P<0.05, ##P<0.01 compared with CKD-MSCs; $P<0.05, $$P<0.01 compared with CKD-MSCs+Con exosomes; AAp<0.01 compared with CKD-MSCs+MT exosomes. FIG. 11C shows a result of Laser Doppler perfusion imaging (LDPI) analysis of the ischemic limbs of CKD mice transplanted with CKD-MSCs treated with exosomes. FIG. 11D shows blood perfusion ratios as analyzed by LDPI (n=10). The values represent mean±SEM, **P<0.01 compared with PBS; ##P<0.01 compared with CKD-MSCs; $P<0.05, $$P<0.01 compared with CKD-MSCs+Con exosomes; AAp<0.01 compared with CKD-MSCs+MT exosomes. FIG. 11E shows representative images illustrating various experimental outcomes (foot necrosis, toe loss, or limb salvage) in ischemic limbs on day 28 after surgery. FIG. 11F shows distribution of different outcomes on postoperative day 28 (n=10). FIGS. 11G and 11H show a result of immunofluorescence staining for CD31 (G; green) and α-SMA (H; red) on postoperative day 28 in ischemic limb tissues. Scale bar=50 Standard quantification of the capillary (FIG. 11G) and arteriole density (FIG. 11H) was calculated as the number of CD31- and α-SMA-positive cells (n=5). The values represent mean±SEM, *P<0.05, **P<0.01 compared with PBS; #P<0.05, ##P<0.01 compared with CKD-MSCs; $$P<0.01 compared with CKD-MSCs+Con exosomes; AAp<0.01 compared with CKD-MSCs+MT exosomes.

### DESCRIPTION OF EXEMPLARY EMBODIMENTS

According to a first embodiment, the present invention provides a pharmaceutical composition for treatment or prevention of chronic kidney disease, comprising exosomes extracted from mesenchymal stem cells derived from a healthy individual co-cultured with melatonin or a culture solution thereof as an active ingredient.

In the pharmaceutical composition according to the present invention, the mesenchymal stem cells are characterized in that they are derived from umbilical cord, umbilical cord blood, bone marrow, fat, muscle, nerve, skin, amniotic membrane, or placenta.

In the pharmaceutical composition according to the present invention, the pharmaceutical composition is characterized in that the pharmaceutical composition increases the expression of a prion protein. For example, the pharmaceutical composition can increase the expression of a prion protein by increasing the expression of miR-4516. More specifically, the pharmaceutical composition can increase the expression of miR-4516, and the increased miR-4516 can increase the expression of a prion protein by downregulating GP78.

In the pharmaceutical composition according to the present invention, the pharmaceutical composition is characterized in that the pharmaceutical composition recovers the mitochondrial functions. For example, the pharmaceutical composition can increase the activities of mitochondrial complex I and complex IV but can decrease SOD2 and ROS.

In the pharmaceutical composition according to the present invention, the pharmaceutical composition is characterized in that the pharmaceutical composition recovers the cellular senescence of mesenchymal stem cells. For example, the pharmaceutical composition can increase the expression of p16, p21, and SMP30.

In the pharmaceutical composition according to the present invention, the pharmaceutical composition is characterized in that the pharmaceutical composition recovers the cell proliferative potential of mesenchymal stem cells. For example, the pharmaceutical composition can increase the expression of p-Akt, p-ERK, p-FAK, and p-Src.

In the pharmaceutical composition according to the present invention, the pharmaceutical composition is characterized in that the pharmaceutical composition increases the expression of proteins associated with angiogenesis of stem cells, anti-inflammation, and cell invasion. For example, the pharmaceutical composition can increase the expression of VEGFR2, VEGFR3, IL-la, MCP-3, I-TAC, MMP-1, MMP-9, and uPAR.

In the pharmaceutical composition according to the present invention, the pharmaceutical composition is characterized in that the pharmaceutical composition is used as an aid for cell treatment. For example, the pharmaceutical composition can be used as an aid for the treatment of cardiovascular disease complications accompanying chronic kidney disease using autologous mesenchymal stem cells derived from a chronic kidney disease (CKD) patient pretreated with the pharmaceutical composition.

In the pharmaceutical composition according to the present invention, the pharmaceutical composition may include a pharmaceutically acceptable carrier, excipient, or diluent, etc. Examples of the pharmaceutically acceptable carrier, excipient, and diluent may include lactose, dextrose, trehalose, sucrose, sorbitol, mannitol, xylitol, erythritol, maltitol, starch, acacia rubber, alginate, gelatin, calcium phosphate, calcium carbonate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, etc., but are not limited thereto. The pharmaceutical composition may be formulated for use, according to the conventional method, in the form of oral administration preparations (e.g., powders, pills, tablets, capsules, suspensions, emulsions, syrups, granules, elixirs, aerosols, etc.), preparations for external use, suppositories, or sterile injectable solutions.

In the pharmaceutical composition according to the present invention, the administration of the pharmaceutical composition refers to the introduction of a predetermined material to a patient in any suitable way, and the route of administration of the pharmaceutical composition can be administered through any general route as long as the drug can reach the target tissue. For example, the pharmaceutical composition can be administered orally or parenterally. The parenteral administration includes transdermal administration, intraperitoneal administration, intravenous administration, intraarterial administration, intramuscular administration, subcutaneous administration, intradermal administration, topical administration, rectal administration, etc. However, the method of parenteral administration is not limited thereto and various administration methods known in the art are not excluded. Additionally, the pharmaceutical composition may be administered by any device capable of transporting an active material to the target tissue or cell.

In the pharmaceutical composition according to the present invention, the preparation for parenteral administration of the pharmaceutical composition may be a sterile aqueous solution, a non-aqueous solvent, a suspension, an emulsion, a lyophilized preparation, or a suppository. The preparation for parenteral administration of the pharmaceutical composition may also be prepared as an injectable preparation. The injectable preparation may be an aqueous injection, non-aqueous injection, aqueous suspension injection, non-aqueous suspension injection, or a solid injection to be dissolved or suspended for use, but the injectable preparation is not limited thereto. The injectable preparation may include at least one kind among distilled water for injection, vegetable oil (e.g., peanut oil, sesame oil, camellia oil, etc.), monoglyceride, diglyceride, propylene glycol, camphor, estradiol benzoate, bismuth subsalicylate, sodium arsenobenzol, or streptomycin sulfate depending on the type of injection, and may optionally include a stabilizer or a preservative.

In the pharmaceutical composition according to the present invention, the pharmaceutical composition may be contained in an amount of about 0.1 wt % to 99 wt %, and preferably about 10 wt % to 90 wt %. Additionally, an appropriate dose of the pharmaceutical composition may be adjusted according to the patient's disease type, disease severity, formulation type, formulation method, patient's age, sex, weight, health status, diet, excretion rate, administration time and administration method. For example, when the pharmaceutical composition is administered, it may be administered one to several times at a daily dose of 0.001 mg/kg to 100 mg/kg.

According to a second embodiment, the present invention provides a method for preparing a pharmaceutical composition for the treatment or prevention of chronic kidney disease (CKD), in which the method includes:
(a) co-culturing mesenchymal stem cells derived from a healthy individual with melatonin;
(b) separating exosomes from the culture solution of Step (a); and
(c) preparing a composition comprising the exosomes separated in Step (b) as an active ingredient.

In the method according to the present invention, the melatonin of Step (a) is contained in a medium at a concentration between 10⁻¹⁰ M or greater and 10⁻⁴ M or less.

In the method according to the present invention, the medium used in co-culture of Step (a) is characterized in that it includes commercially prepared media or artificially synthesized, such as Dulbecco's Modified Eagle's Medium (DMEM), Minimal Essential Medium (MEM), Basal Medium Eagle (BME), RPMI1640, Dulbecco's Modified Eagle's Medium: Nutrient Mixture F-10 (DMEM/F-10), Dulbecco's Modified Eagle's Medium: Nutrient Mixture F-12 (DMEM/F-12), α-Minimal essential Medium (α-MEM), Glasgow's Minimal Essential Medium (G-MEM), Isocove's Modified Dulbecco's Medium (IMDM), KnockOut DMEM, etc.

In the method according to the present invention, the separation of Step (b) is characterized in that the separation of Step (b) is performed by centrifugation, ultracentrifugation, filtration by a filter, gel filtration chromatography, free-flow electrophoresis, capillary electrophoresis, separation using a polymer, and a combination thereof.

In the method according to the present invention, the exosomes of Step (b) are characterized in that they employ centrifugation. The centrifugation may be performed at 5,000-500,000 g for 10 minutes to 5 hours.

According to a third embodiment, the present invention provides a method for promoting the proliferation of mesenchymal stem cells derived from a chronic kidney disease (CKD) patient or increasing the survival rate of the patient, wherein the method is characterized in that it includes:
(a) co-culturing mesenchymal stem cells derived from a healthy individual with melatonin;
(b) separating exosomes from the culture solution of Step (a); and
(c) treating the exosomes separated in Step (b) to autologous stem cells derived from a chronic kidney disease patient of a mammal excluding humans and increasing the expression of a prion protein.

In the method according to the present invention, the melatonin of Step (a) is contained in a medium at a concentration between 10⁻¹⁰ M or greater and 10⁻⁴ M or less.

In the method according to the present invention, the medium used in co-culture of Step (a) is characterized in that it includes commercially prepared media or artificially synthesized, such as Dulbecco's Modified Eagle's Medium (DMEM), Minimal Essential Medium (MEM), Basal Medium Eagle (BME), RPMI1640, Dulbecco's Modified Eagle's Medium: Nutrient Mixture F-10 (DMEM/F-10), Dulbecco's Modified Eagle's Medium: Nutrient Mixture F-12 (DMEM/F-12), α-Minimal essential Medium (α-MEM), Glasgow's Minimal Essential Medium (G-MEM), Isocove's Modified Dulbecco's Medium (IMDM), KnockOut DMEM, etc.

In the method according to the present invention, the separation of Step (b) is characterized in that the separation of Step (b) is performed by centrifugation, ultracentrifugation, filtration by a filter, gel filtration chromatography, free-flow electrophoresis, capillary electrophoresis, separation using a polymer, and a combination thereof.

In the method according to the present invention, the exosomes of Step (b) are characterized in that they employ centrifugation. The centrifugation may be performed at 5,000-500,000 g for 10 minutes to 5 hours.

In the method according to the present invention, the mesenchymal stem cells are characterized in that they are derived from umbilical cord, umbilical cord blood, bone marrow, fat, muscle, nerve, skin, amniotic membrane, or placenta.

In the method according to the present invention, the exosomes isolated from mesenchymal stem cells derived from a healthy individual are characterized in that they increase the expression of miR-4516, and the increased miR-4516 increases the expression of a prion protein by downregulating GP78.

In the method according to the present invention, the exosomes isolated from mesenchymal stem cells derived from a healthy individual are characterized in that they recover mitochondrial functions, cellular senescence, and cell proliferative potential.

In the method according to the present invention, the exosomes isolated from mesenchymal stem cells derived from a healthy individual are characterized in that they increase the expression of proteins associated with angiogenesis of mesenchymal stem cells, anti-inflammation, and cell invasion.

Hereinafter, the present invention will be described in detail by Examples. However, the following examples are merely illustrative of the present invention, and the contents of the present invention are not limited by the following Examples.

<Materials and Method>

1. Serum Samples

The local ethics committee approved this study, and informed consent was obtained from all the individuals. Explanted sera (n=37) were obtained from patients with CKD at the Seoul National University Hospital in Seoul, Korea. Upon fulfilling transplantation criteria, the control samples were obtained from healthy patients (n=40) at the National Cancer Center in Seoul, Korea. Chronic kidney disease diagnoses were made based on abnormal kidney function with an estimated glomerular filtration rate (eGFR) <25 mL/min/1.73 m2 over 3 months.

2. Culturing Healthy and CKD-MSCs

Human adipose tissue-derived MSCs were isolated from one healthy individual (healthy MSCs) and one patient with CKD (CKD-MSCs) from the Soonchunhyang University Seoul Hospital. Chronic kidney disease was diagnosed in a patient with impaired kidney function and an estimated eGFR<35 mL/(min·1.73 m2) for more than 3 months (stage 3b). Both types of isolates were positive for the MSC surface markers CD44 and Sca-1 and negative for CD45 and CD11b.10. They were also differentiated into chondrogenic, adipogenic, and osteogenic cells under specific media conditions. Ten healthy and CKD-MSCs were transferred to α-Minimum Essential Medium (α-MEM; Gibco BRL) containing 10% (v/v) fetal bovine serum (FBS; Gibco BRL) and 100 U/mL penicillin/streptomycin (Gibco BRL) within 3 days and grown in a humidified 5% CO2 incubator at 37° C.

3. Isolation of MSC-Derived Exosomes

Exosomes from healthy and CKD-MSCs were extracted using an exosome isolation kit (Rosetta Exosome) and concentrated using centrifugal filters (Millipore).

4. Treatment of CKD-MSCs with Exosomes Derived from Healthy MSCs

CKD-MSCs were treated with 30 μg of exosomes derived from healthy MSCs for 24 hours. The concentration of exosomes was assessed using colorimetric BCA assay (Thermo Fisher Scientific).

5. Quantification of microRNA (miRNA)

Healthy MSCs and their respective exosome preparations were used to extract total RNA (DNase digested) with the miRNeasy Mini Kit (Qiagen). Quantitative real-time polymerase chain reaction (qRT-PCR) was performed using the TaqMan Small RNA Assay (Thermo Fisher Scientific) to determine the expression of miRNAs normalized to U6 rRNA or β-actin.

6. SOD2 Activity

Total protein was extracted from CKD-MSCs using a RIPA extraction buffer (Thermo Fisher Scientific). SOD2 activity was measured using a SOD activity kit (Enzo) as per the kit instructions. To inhibit SOD1 activity, 40 μg of a protein containing 2 mmol/L cyanide ion was added to each well. The absorbance was measured at 450 nm every minute for 15 minutes using a microplate reader (BMG Labtech), and activity was calculated according to the kit manual.

7. Western Blot Analysis

Whole cell, cytosol, and mitochondrial fraction lysates were prepared from healthy and CKD-MSCs. These samples (30 μg of protein each) were analyzed by sodium dodecyl sulfate-polyacrylamide gel electrophoresis using gels with porosity between 8% and 12% followed by transfer to a nitrocellulose membrane. After the blots were washed with TBST (10 mmol/L Tris-HCl [pH 7.6], 150 mmol/L NaCl, 0.05% Tween 20), they were blocked with 5% skim milk for 1 hour at room temperature followed by incubation with the following primary antibodies: p16 (Clone No. D7C1M; Cat. No. #80772; Cell Signaling Technology), p21 (Clone No. WA-1; Cat. No. sc51689; Santa Cruz Biotechnology), SMP30 (Clone No. 17; Cat. No. sc-130344; Santa Cruz Biotechnology), p-Akt (Ser 473; Cat. No. sc-101629; Santa Cruz Biotechnology), Akt (Clone No. 281046; Cat. No. MAB2055; R&D systems), p-ERK (Clone No. E-4; Cat. No. sc-7383; Santa Cruz Biotechnology), ERK (Clone No. 216703; Cat. No. MAB1576; R&D systems), p-FAK (Tyr 576-R; Cat. No. sc-16563-R; Santa Cruz Biotechnology), FAK (Clone No. OTI4D11; Cat. No. NBP2-45923), p-Src (Clone No. E-4; Cat. No. sc-7383; Santa Cruz Biotechnology), Src (Clone No. 327 537; Cat. No. MAB3389; R&D systems), CD81 (Clone No. B-11; Cat. No. sc-166029; Santa Cruz Biotechnology), CD63 (Clone No. MX-49.129.5; Cat. No. sc-5275; Santa Cruz Biotechnology), GP78 (Clone No. F-3; Cat. No. sc-166358; Santa Cruz Biotechnology), PrPC (Clone No. H8; Cat. No. sc393165; Santa Cruz Biotechnology), ubiquitin (Clone No. Ubi1; Cat. No. NB300-130; NOVUS, Littleton), and β-actin (Santa Cruz Biotechnology). The membranes were then washed and incubated with the respective goat anti-rabbit IgG or goat antimouse IgG secondary antibodies (Santa Cruz Biotechnology). The blots were developed using enhanced chemiluminescence (Amersham Pharmacia Biotech).

8. Senescence β-Galactosidase (SA-β-Gal) Cell Staining

Healthy and CKD-MSCs (with or without melatonin treatment) were cultured in 24-well plates (5000 cells/well) and assayed using the Senescence β-Galactosidase Staining kit (Cell Signaling Technology) following the kit protocol. Development of blue color was observed by light microscopy (Olympus).

9. Single-Cell Expansion Assay

Healthy and CKD-MSC suspensions containing 103 cells in 10 mL complete medium were diluted 1:10 (cells:complete medium), and 100 μL of the dilutions (about 1 cell/100 μL) was seeded into a 96-well plate. The cells were cultured upon treatment with PBS, Con exosomes, MT exosomes, MT exosomes+siPRNP, and MT exosomes+siScr in a humidified incubator. Each well was examined for growth on day 10.

10. Kinase Assays for Complex I and IV Activity

Protein lysates (30-50 μg) were assayed for the activity of complex I and IV (Abcam). Activation of complex I and IV was quantified by measuring absorbance at 450 nm on a microplate reader (BMG Labtech).

11. Human Angiogenesis Protein Array

Levels of angiogenesis-associated proteins in CKD-MSCs treated with PBS, Con exosomes, and MT exosomes were determined using the Human Angiogenesis Antibody Array (Abcam). Total protein lysates (200 μg) were suspended in bovine serum albumin (blocking buffer provided) and assayed as per the kit protocol.

12. Murine Hindlimb Ischemia Model with CKD

Eight-week-old male BALB/c nude mice were fed an adenine-containing diet (0.75% adenine) for 1-2 weeks, and body weights were measured weekly. The mice were randomly distributed to four groups consisting of 10 mice each. Blood was stored at −80° C. for measuring blood urea nitrogen and creatinine posteuthanasia. To understand vascular disease and assess angiogenesis in CKD, a murine hind limb ischemia model with CKD was established after adenine-loaded feeding for 1 week. Ischemia was induced by ligation and excision of the proximal femoral artery and boundary vessels of the CKD mice. Within 6 hours of the surgical procedure, cells were injected into ischemic sites (106 cells/100 μL of PBS/mouse; single injection; 5 mice/treatment group) of the CKD mice. Blood perfusion was calculated by the ratio of blood flow in the ischemic (left) limb to that in the nonischemic (right) limb on postoperative days 0, 7, 14, 21, and 28 using laser Doppler perfusion imaging (LDPI; Moor Instruments).

13. Immunohistochemical Staining

Ischemic thigh tissues were removed on postoperative days 3 and 28, fixed with 4% paraformaldehyde (Sigma), and each tissue sample was embedded in paraffin. For measuring apoptosis and proliferation, the tissues were stained for TUNEL (Trevigen) and PCNA (Santa Cruz Biotechnology), respectively. Immunofluorescence staining was performed with primary antibodies against CD11b (Abcam), CD31 (Santa Cruz Biotechnology), and α-SMA (alpha-smooth muscle actin; Santa Cruz Biotechnology) followed by secondary antibodies conjugated with Alexa Fluor 488 or 594 (Thermo Fisher Scientific). Nuclei were stained with 4′,6-diamidino-2-phenylindole (Sigma), and the samples were examined by confocal microscopy (Olympus).

14. Statistical Analysis

Two-tailed Student's t test and one- or two-way analysis of variance were used to calculate significance between groups, and the results were expressed as standard error of mean (SEM). Comparisons between three or more groups were made using Dunnett's or Tukey's post hoc test. Data were considered significantly different at P<0.05.

### EXAMPLES

### Example 1. Verification of Kidney Function of Chronic Kidney Disease (CKD) Patient

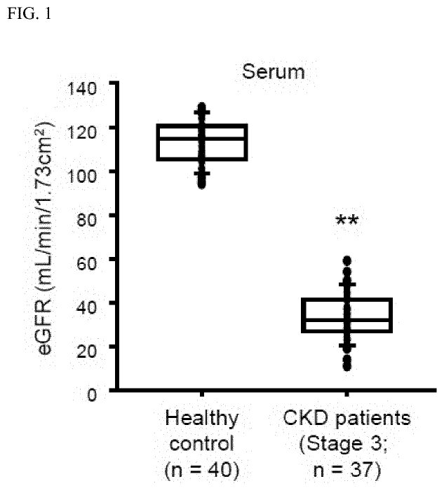

In order to compare the kidney functions of a chronic kidney disease (CKD) patient with that of a healthy individual, the glomerular filtration rate (GFR) of the healthy individual and the chronic kidney disease (CKD) patient was qauntified (eGFR=175*Serum Creatinine-1.154*Age-0.203*[1.210 if Blank]*[0.742 if Female]) and the results are shown in FIG. 1.

### Example 2. Confirmation of Weakening of Stem Cell Potential of Mesenchymal Stem Cells Derived from Chronic Kidney Disease (CKD) Patient

In order to confirm the stem cell potential of mesenchymal stem cells derived from a chronic kidney disease (CKD) patient, β-galactosidase staining for the stem cells derived from a healthy individual and a chronic kidney disease (CKD) patient, respectively; Western blot for the senescence-associated proteins (i.e., p16, p21, and SMP30) in stem cells of each group; single-cell expansion assay for the confirmation of proliferative potential in stem cells of each group; bromodeoxyuridine/5-bromo-2′-deoxyuridine (BrdU) DNA staining for the confirmation of the level of proliferation in stem cells of each group; measurement of S phase (division stage) for stem cells of each group; and Western blot for p-Akt, p-ERK, p-FAK, and p-Src for the confirmation of activation of cellular functions in stem cells of each group were performed.

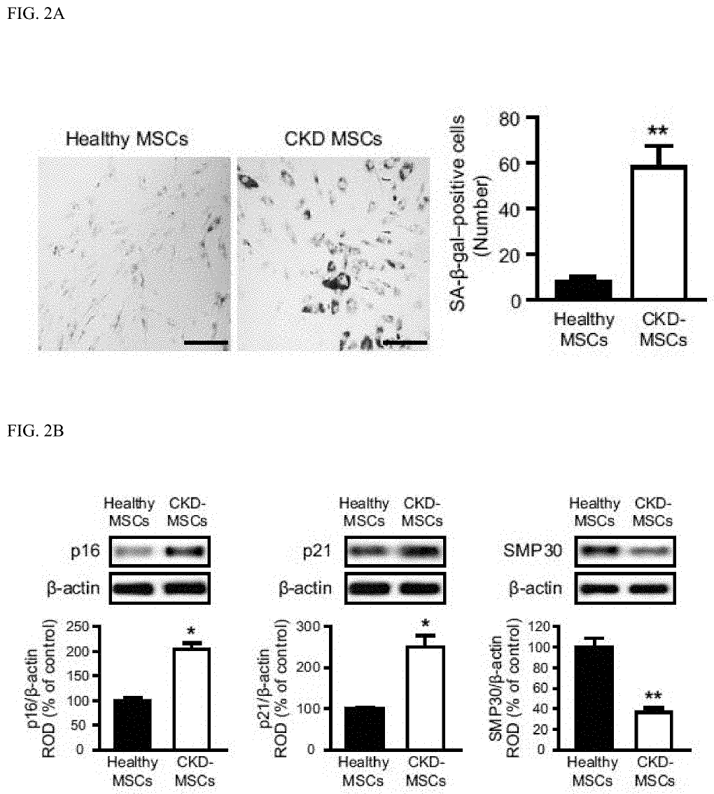

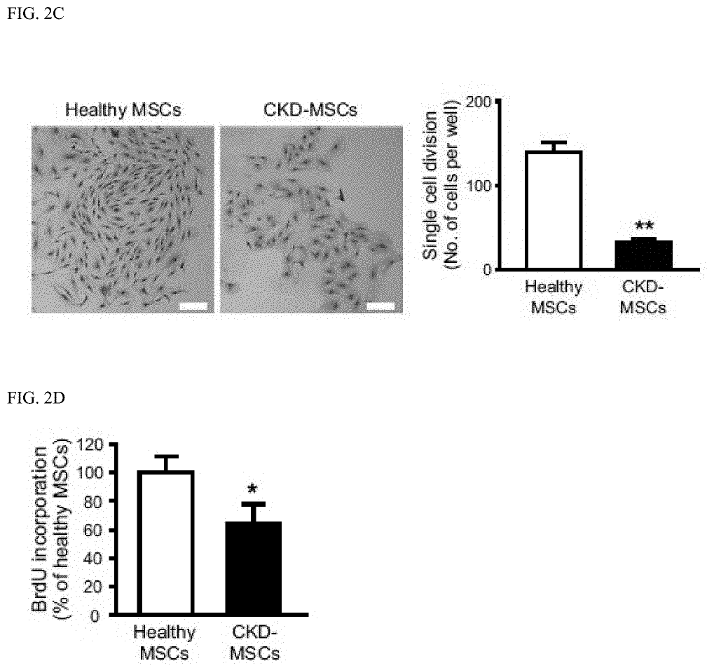

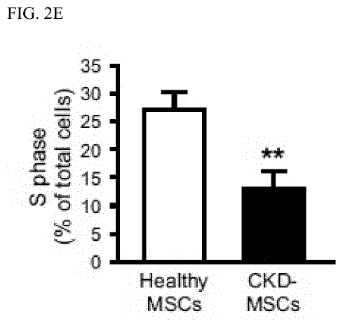

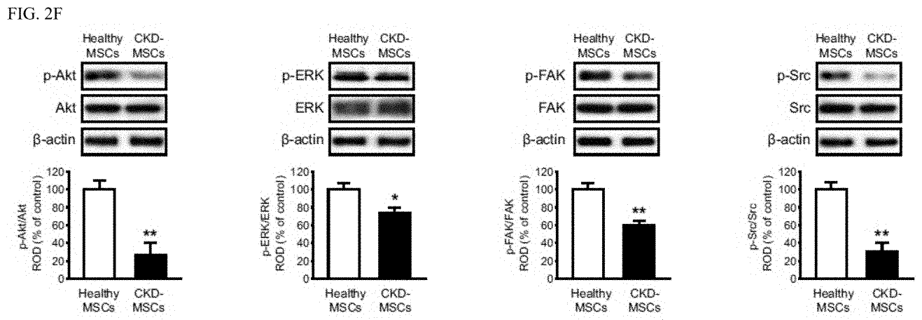

As a result, it was shown that in the case of the mesenchymal stem cells derived from a chronic kidney disease (CKD) patient, senescence was progressed rapidly (FIG. 2A), and the expression of the senescence-associated proteins (i.e., p16, p21, and SMP30) was increased (FIG. 2B). In the mesenchymal stem cells derived from a chronic kidney disease (CKD) patient, it was confirmed that the single cell proliferative potential was reduced (FIG. 2C-D), and S phase (cell division stage) was decreased (FIG. 2E). Additionally, it was confirmed that the expression of cellular function activating proteins (i.e., p-Akt, p-ERK, p-FAK, and p-Src) was decreased in the mesenchymal stem cells derived from a chronic kidney disease (CKD) patient (FIG. 2F).

### Example 3. Evaluation of Exosomes Extracted from Mesenchymal Stem Cells Derived from a Healthy Individual after Treatment with Melatonin

After the melatonin treatment (1 μM, 24 hours), the presence of expression of CD81 and CD63 (i.e., the indicator proteins of exosomes) in the exosomes extracted from the mesenchymal stem cells derived from a healthy individual was observed by Western blot and Cryogenic transmission electron microscopy (cryo-TEM), and the exosomes of each group were measured by attaching the antibodies of CD81 and CD63 (i.e., the indicator proteins of exosomes), and the size of the exosomes was measured by the nanoparticle tracking analysis (NTA).

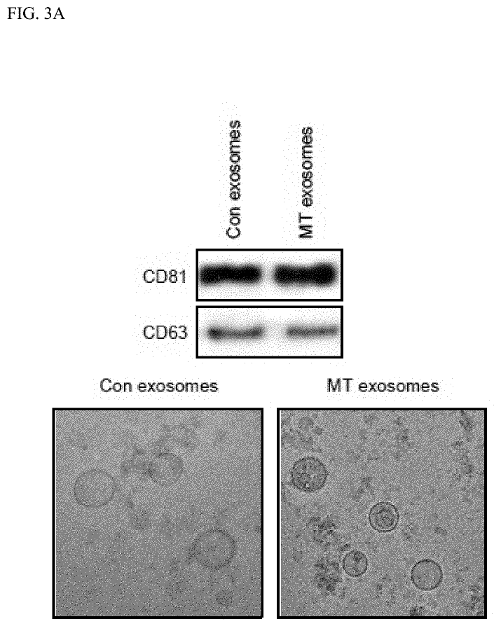

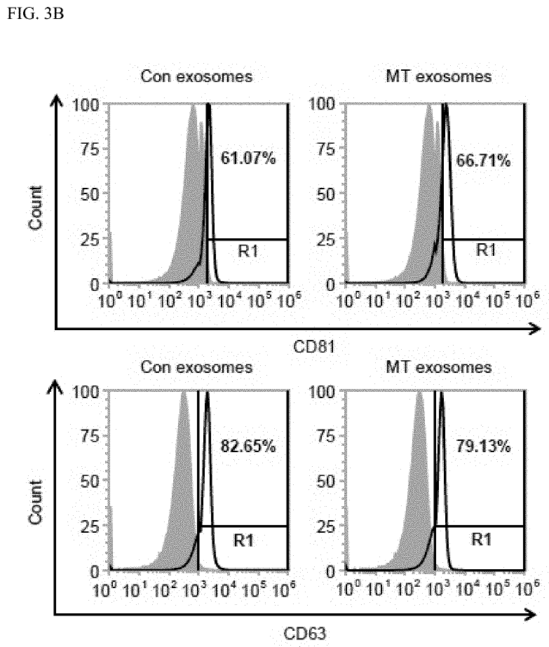

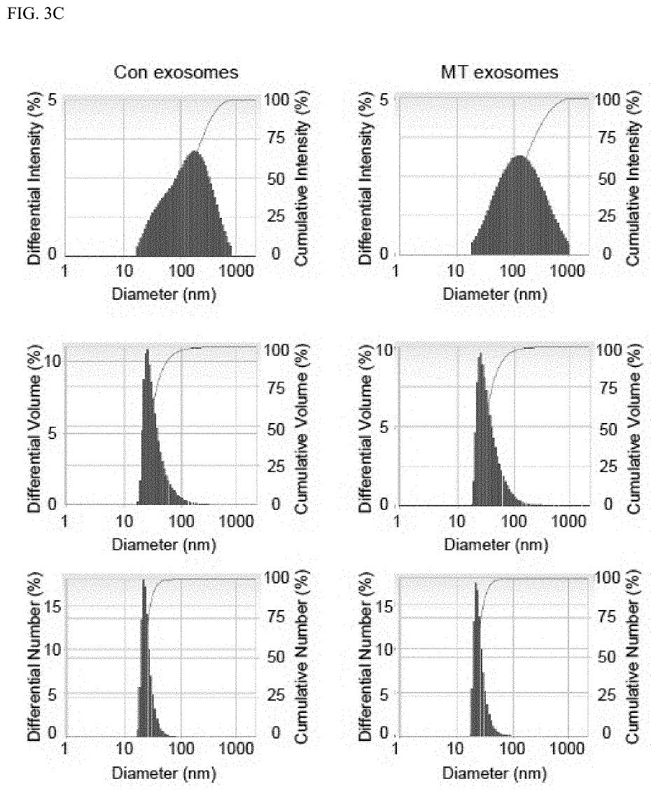

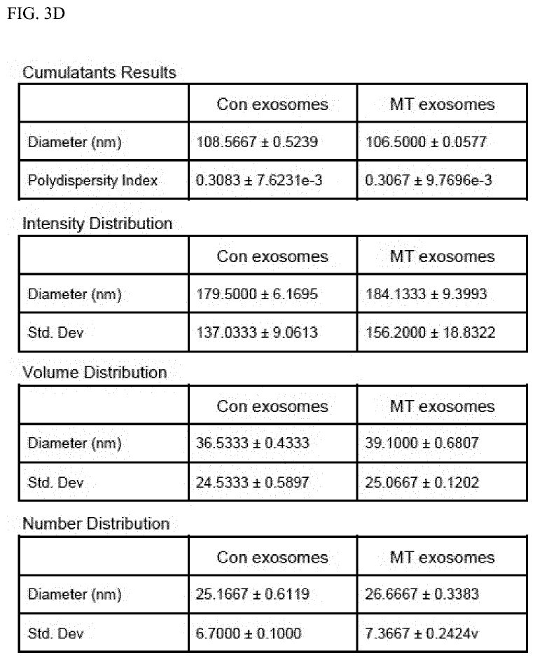

As a result, it was confirmed that after melatonin treatment, the exosomes extracted from the mesenchymal stem cells derived from a healthy individual were the exosomes suitable for international standards (FIGS. 3A-D).

### Example 4. Confirmation of Main Factors in Exosomes Extracted from the Mesenchymal Stem Cells Derived from a Healthy Individual after Melatonin Treatment

The presence of expression of prion proteins present in the sera of a healthy individual and a chronic kidney disease (CKD) patient was confirmed by Enzyme Linked Immuno Sorbent Assay (ELISA); and then, the expression of prion proteins was measured in mesenchymal stem cells derived from a healthy individual and a chronic kidney disease (CKD) patient and in exosomes derived from stem cells by ELISA; and the expression of prion proteins was measured in mesenchymal stem cells derived from a chronic kidney disease (CKD) patient after treatment with melatonin, exosomes extracted from mesenchymal stem cells derived from a healthy individual (Con exosome), and exosomes extracted from stem cells derived from a melatonin-treated healthy individual (MT exosomes) by ELISA. Additionally, the expression level of micro RNA (miR) in exosomes extracted from mesenchymal stem cells derived from a melatonin-treated healthy individual was compared with that in exosomes not treated with melatonin by RNA microarray, and the expression of miR-4516 in exosomes of the above group was confirmed by RNA microarray, and the expression level of miR-4516 in mesenchymal stem cells derived from a melatonin-treated healthy individual and a chronic kidney disease (CKD) patient was confirmed by quantitative real-time polymerase chain reaction (qPCR). The level of prion proteins in exosomes extracted from stem cells derived from a melatonin-treated healthy individual after pretreatment with a miR-4516 inhibitor was measured by ELISA, and the exosomes extracted from stem cells derived from a healthy individual, and the exosomes extracted from stem cells derived from a melatonin-treated healthy individual, and the exosomes extracted from stem cells derived from a melatonin-treated healthy individual after pretreatment with a miR-4516 inhibitor were each treated on stem cells derived from a chronic kidney disease (CKD) patient, and the expression of prion proteins in the cells was measured by ELISA.

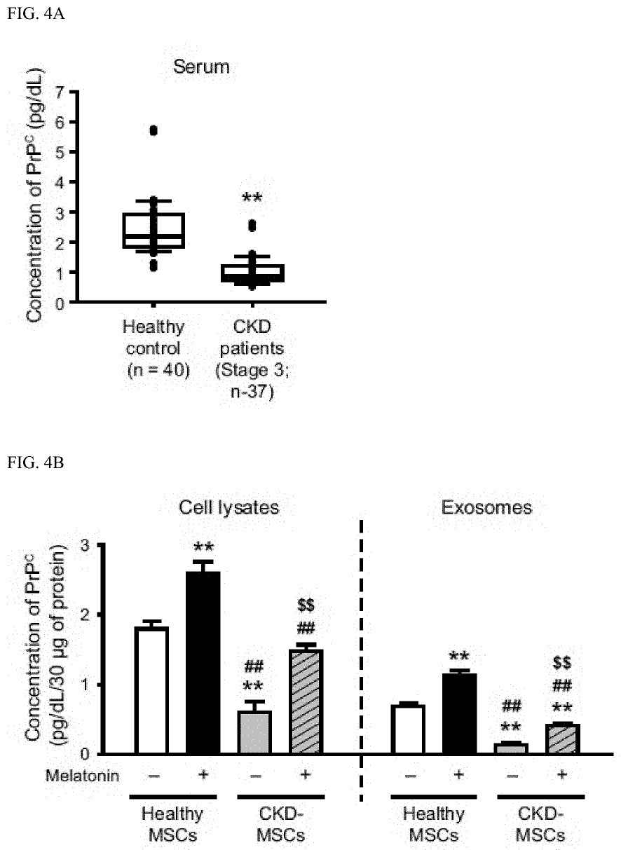

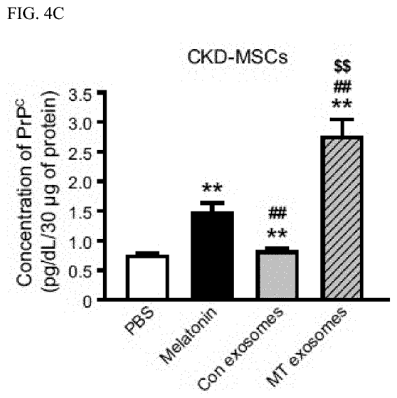

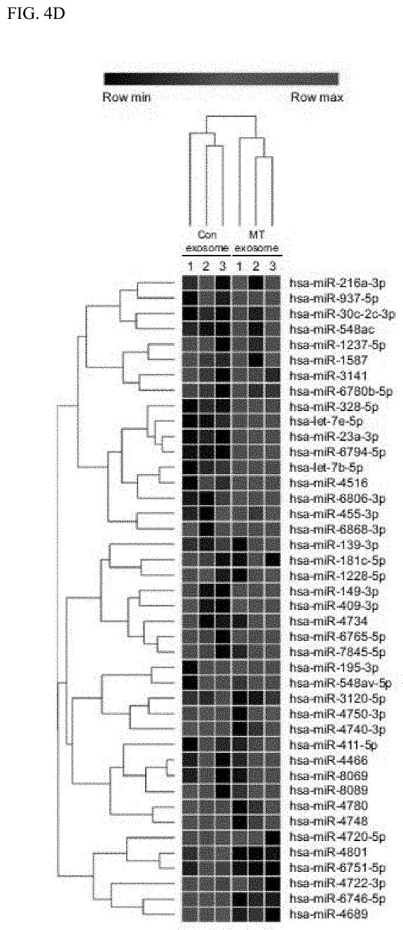

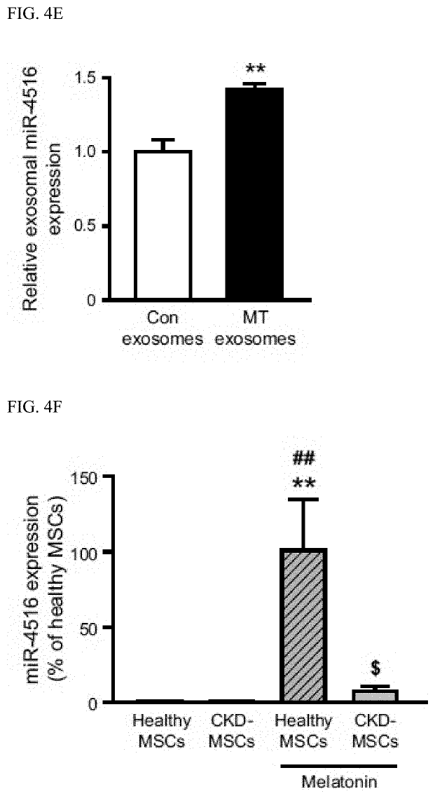

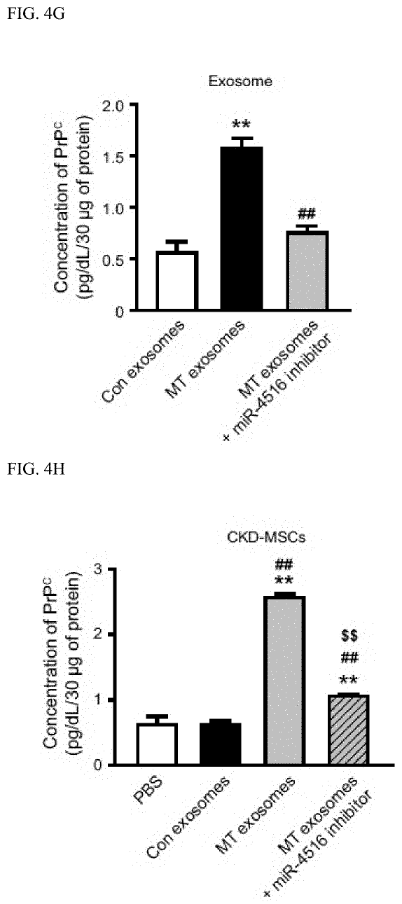

As a result, the expression of prion proteins present in the serum of a chronic kidney disease (CKD) patient was shown to decrease, whereas the expression of prion proteins present in the exosomes extracted from stem cells after treatment of the mesenchymal stem cells derived from a healthy individual and a chronic kidney disease (CKD) patient with melatonin was shown to increase (FIGS. 4B and 4C). In particular, comparing the expression level of miR in Con exosome and in MT exosome, the expression level of miR was shown to increase in MT exosome (FIGS. 4D and 4E), and the expression level of miR-4516 was shown to increase in each group of stem cells treated with melatonin (FIG. 4F). Meanwhile, the expression of prion proteins in exosomes extracted from mesenchymal stem cells derived from a healthy individual was shown to decrease even when pretreated with a miR-4516 inhibitor (FIG. 4G). Additionally, the expression of prion proteins was shown to be highest in stem cells derived from a chronic kidney disease (CKD) patient with MT exosome, whereas the expression of prion proteins was shown to decrease in the exosomes in which a miR-4516 inhibitor is included was shown (FIG. 4H).

### Example 5. Confirmation of Whether miR-4516 is Core Factor in Regulation of Prion Protein

In a case where the expression of miR-4516 is inhibited or enhanced, the expression of miR-4516 in exosomes extracted from autologous mesenchymal stem cells derived from a healthy individual was confirmed by quantitative real time polymerase chain reaction (qPCR), and the expression of prion proteins in exosomes extracted from autologous mesenchymal stem cells derived from a healthy individual and a chronic kidney disease (CKD) patient treated with melatonin alone, the expression of prion proteins in exosomes extracted from stem cells by inhibition or enhancement of the expression of miR-4516, and in exosomes extracted from a healthy individual were each measured by ELISA.

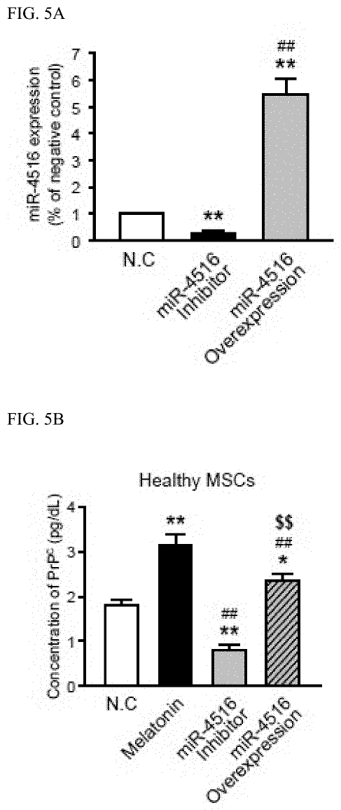

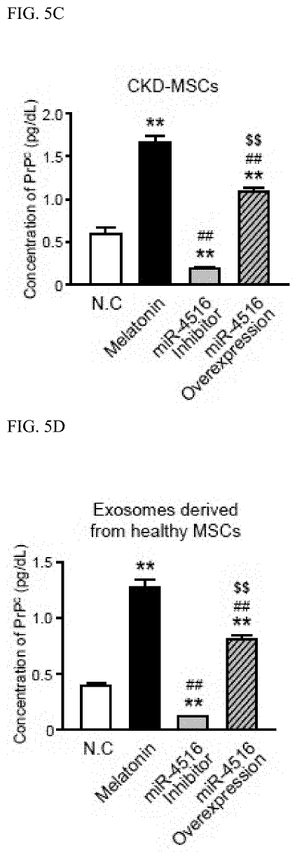

As a result, it was confirmed that the expression of prion proteins changed significantly in exosomes extracted from stem cells derived from a healthy individual, exosomes extracted from stem cells derived from a chronic kidney disease (CKD) patient, and exosomes extracted from stem cells derived from a healthy individual according to the expression level of miR-4516 (FIGS. 5A and 5B).

Additionally, in a case where the expression of miR-4516 is inhibited or enhanced in autologous mesenchymal stem cells derived from a healthy individual, the expression of GP78 (i.e., E3 ligase) was confirmed via Western blot, and the degree of protein degradation and the expression of prion proteins by ubiquitin were measured via Western blot by inhibiting or enhancing the expression of miR-4516, and the expressions of GP78 (i.e., E3 ligase) and prion proteins when exosomes, which were extracted by treating the autologous mesenchymal stem cells derived from a healthy individual with melatonin after inhibition of miR-4516 expression, were treated on mesenchymal stem cells derived from a chronic kidney disease (CKD) patient were measured by Western blot.

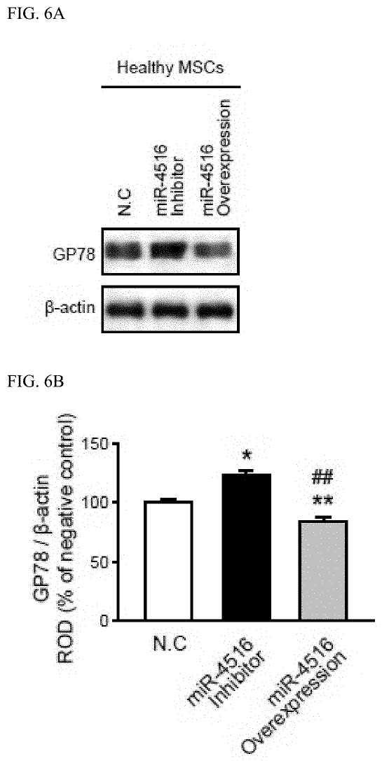

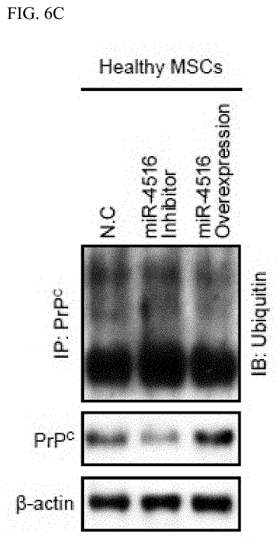

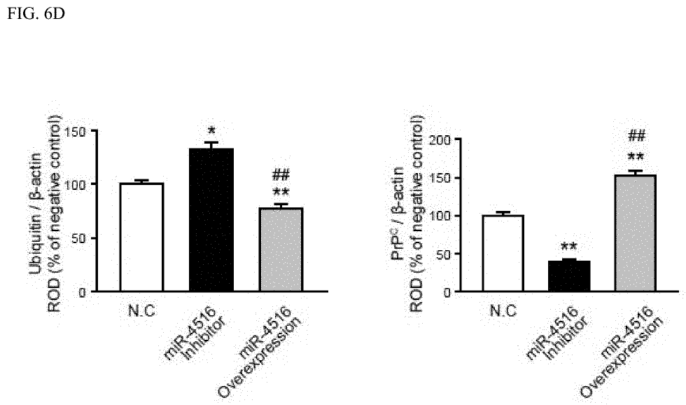

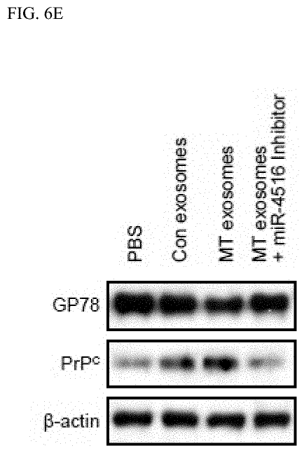

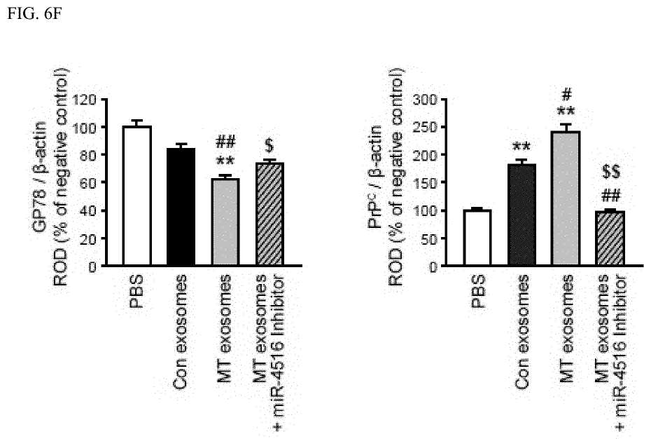

As a result, it was confirmed that the expression of GP78 (i.e., protease E3 ligase) in the mesenchymal stem cells derived from a healthy individual was significantly changed according to the expression level of miR-4516 (FIGS. 6A and 6B), and the conjugation between prion proteins and ubiquitin in the mesenchymal stem cells derived from a healthy individual was significantly changed according to the expression level of miR-4516 (FIGS. 6C and 6D), and additionally, the expression levels of GP78 and prion proteins in stem cells derived from a chronic kidney disease (CKD) patient were shown to change according to the expression level of miR-4516 in exosomes (FIGS. 6E and 6F).

### Example 6. Improvement of Function in Mesenchymal Stem Cells Derived from a Chronic Kidney Disease (CKD) Patient Due to Treatment with Melatonin Exosomes

6-1. Confirmation of Improvement of Mitochondrial Function in Stem Cells Derived from a Chronic Kidney Disease (CKD) Patient Using Melatonin Exosomes

Normal and abnormal mitochondria in mesenchymal stem cells derived from a chronic kidney disease (CKD) patient treated with the pharmaceutical composition according to the present invention containing the melatonin-treated exosomes were confirmed by an electron microscope, and the enzyme activities of mitochondrial Complex I and Complex IV in mesenchymal stem cells derived from a chronic kidney disease (CKD) patient treated with the pharmaceutical composition was confirmed by ELISA, and the enzyme activity of SOD2 in mesenchymal stem cells derived from a chronic kidney disease (CKD) patient treated with the pharmaceutical composition was confirmed by colorimetric activity, and the level of ROS present in the mitochondria of mesenchymal stem cells derived from a chronic kidney disease (CKD) patient treated with the pharmaceutical composition was confirmed via fluorescence-activated cell sorting (FACS).

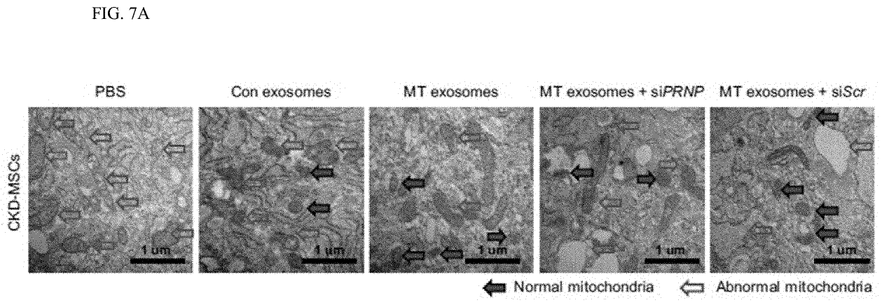

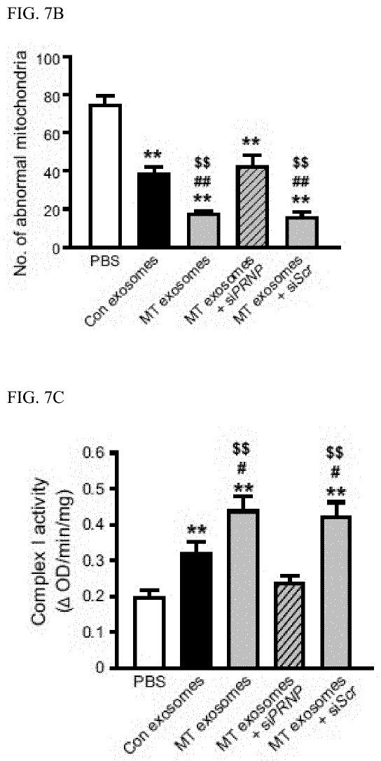

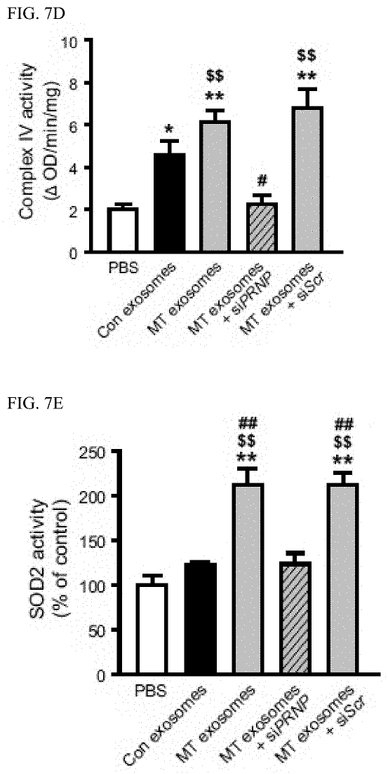

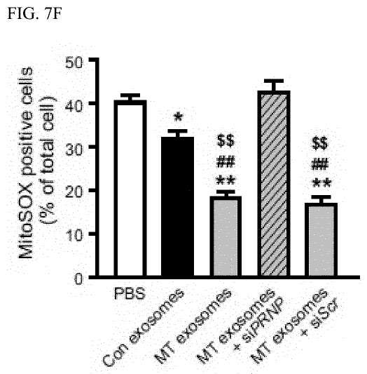

As a result, while the exosomes extracted from the mesenchymal stem cells derived from a healthy individual treated with melatonin reduced abnormal mitochondria, the exosomes extracted from the mesenchymal stem cells derived from a healthy individual treated with melatonin after inhibition of the expression of prion proteins using PRNP siRNA was shown not to recover the mesenchymal stem cells derived from a chronic kidney disease (CKD) patient (FIGS. 7A and 7B). While the exosomes extracted from the mesenchymal stem cells derived from a healthy individual exosome treated with melatonin increased the activities of Complex I & IV (i.e., mitochondrial electron transport system enzymes) in the mesenchymal stem cells derived from a chronic kidney disease (CKD) patient, the exosomes extracted from the mesenchymal stem cells derived from a healthy individual treated with melatonin after inhibition of the expression of prion proteins using PRNP siRNA were shown to decrease the activities of Complex I & IV (i.e., mitochondrial electron transport system enzymes) in the mesenchymal stem cells derived from a chronic kidney disease (CKD) patient (FIGS. 7C and 7D). While the exosomes extracted from the mesenchymal stem cells derived from a healthy individual treated with melatonin were shown to increase the activity of superoxide dismutase (SOD) in the mitochondria of the mesenchymal stem cells derived from a chronic kidney disease (CKD) patient, the exosomes extracted from the mesenchymal stem cells derived from a healthy individual treated with melatonin after inhibition of the expression of prion proteins using PRNP siRNA were shown to decrease the activity of superoxide dismutase (SOD) in the mitochondria of the mesenchymal stem cells derived from a chronic kidney disease (CKD) patient (FIG. 7E). Additionally, while the exosomes extracted from the mesenchymal stem cells derived from a healthy individual treated with melatonin decreased the amount of ROS in the mitochondria of the mesenchymal stem cells derived from a chronic kidney disease (CKD) patient, the exosomes extracted from the mesenchymal stem cells derived from a healthy individual treated with melatonin after inhibition of the expression of prion proteins using PRNP siRNA were shown to increase the amount of ROS in the mitochondria of the mesenchymal stem cells derived from a chronic kidney disease (CKD) patient (FIG. 7F).

6-2. Confirmation of Cellular Senescence of Stem Cells Derived from a Chronic Kidney Disease (CKD) Patient Using Melatonin Exosomes

The senescence of autologous mesenchymal stem cells derived from a chronic kidney disease (CKD) patient pretreated with the pharmaceutical composition according to the present invention was confirmed through beta galactosidase staining, and the expressions of senescence-associated proteins (i.e., p16, p21, and SMP30) of autologous mesenchymal stem cells derived from a chronic kidney disease (CKD) patient pretreated with the pharmaceutical composition were confirmed by western blot.

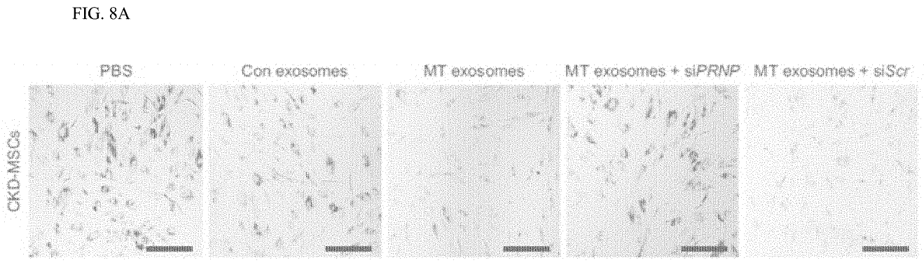

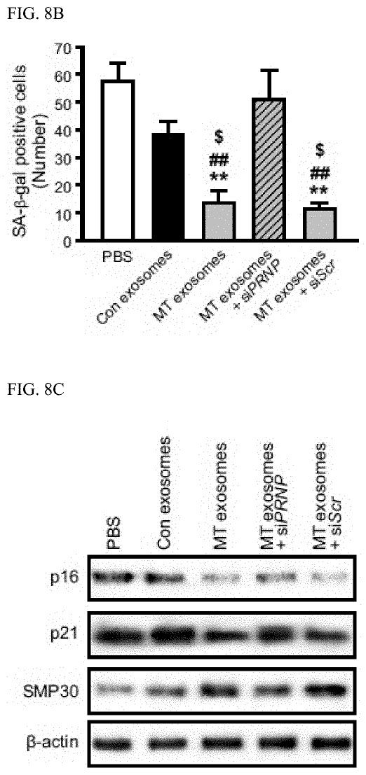

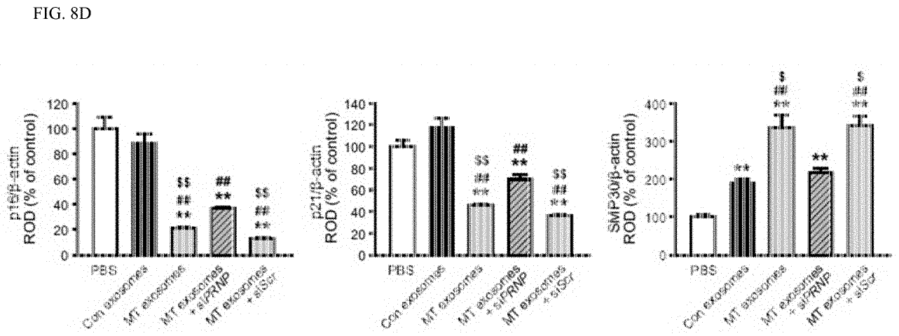

As a result, while the exosomes extracted from the mesenchymal stem cells derived from a healthy individual treated with melatonin inhibited senescence of the mesenchymal stem cells derived from a chronic kidney disease (CKD) patient, the exosomes extracted from the mesenchymal stem cells derived from a healthy individual treated with melatonin after inhibition of the expression of prion proteins using PRNP siRNA were shown not to inhibit the senescence of the mesenchymal stem cells derived from a chronic kidney disease (CKD) patient (FIGS. 8A and 8B). Additionally, while the exosomes extracted from the mesenchymal stem cells derived from a healthy individual treated with melatonin decreased the expression of the senescence-associated proteins (i.e., p16, p21, and SMP30) of the mesenchymal stem cells derived from a chronic kidney disease (CKD) patient, the exosomes extracted from the mesenchymal stem cells derived from a healthy individual treated with melatonin after inhibition of the expression of prion proteins using PRNP siRNA were shown to increase the expression of the senescence-associated proteins of the mesenchymal stem cells derived from a chronic kidney disease (CKD) patient (FIGS. 8C and 8D).

6-3. Confirmation of Stem Cell Proliferative Potential of Stem Cells Derived from a Chronic Kidney Disease (CKD) Patient Using Melatonin Exosomes

The proliferation of the autologous mesenchymal stem cells derived from a chronic kidney disease (CKD) patient pretreated with the pharmaceutical composition according to the present invention was measured through a single cell proliferation method, and the expressions of p-Akt, p-ERK, p-FAK, and p-Src of the autologous mesenchymal stem cells derived from a chronic kidney disease (CKD) patient pretreated with the pharmaceutical composition were measured by Western blot.

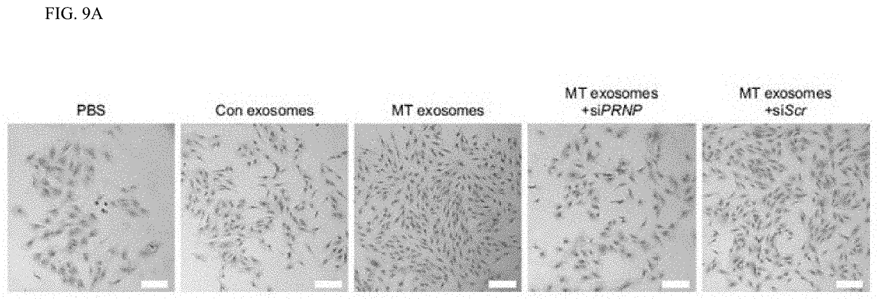

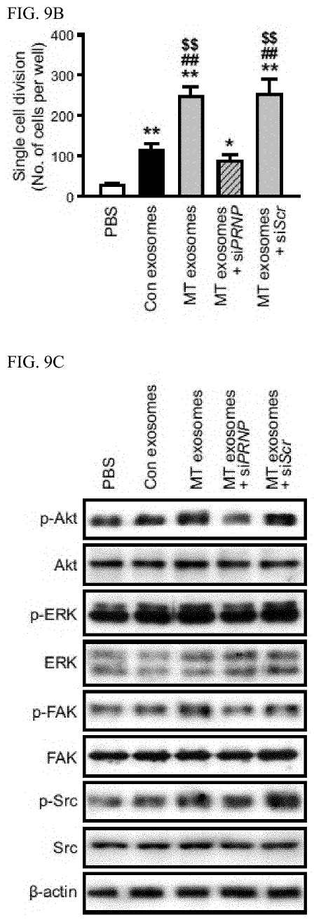

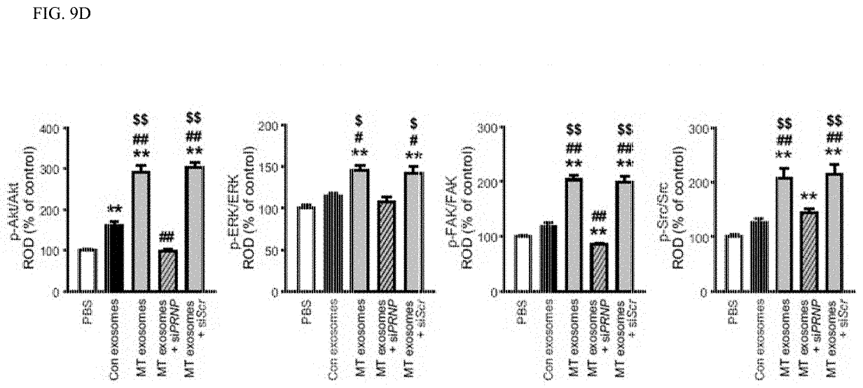

As a result, while the exosomes extracted from the mesenchymal stem cells derived from a healthy individual treated with melatonin enhanced the proliferative potential of the mesenchymal stem cells derived from a chronic kidney disease (CKD) patient, the exosomes extracted from the mesenchymal stem cells derived from a healthy individual treated with melatonin after inhibition of the expression of prion proteins using PRNP siRNA were shown not to enhance the proliferative potential of the mesenchymal stem cells derived from a chronic kidney disease (CKD) patient (FIGS. 9A and 9B). Additionally, while the exosomes extracted from the mesenchymal stem cells derived from a healthy individual treated with melatonin increased the expressions of p-Akt, p-ERK, p-FAK, and p-Src (i.e., cellular function activating proteins) of the mesenchymal stem cells derived from a chronic kidney disease (CKD) patient, the exosomes extracted from the mesenchymal stem cells derived from a healthy individual treated with melatonin after inhibition of the expression of prion proteins using PRNP siRNA were shown to decrease the expressions of cellular function activating proteins of the mesenchymal stem cells derived from a chronic kidney disease (CKD) patient (FIGS. 9C and 9D).

Confirmation of Angiogenesis Potential of Stem Cells Derived from a Chronic Kidney Disease (CKD) Patient Using Melatonin Exosomes

The expression levels of angiogenesis-associated proteins of the autologous mesenchymal stem cells derived from a chronic kidney disease (CKD) patient pretreated with the pharmaceutical composition according to the present invention were confirmed by the angiogenesis antibody array-membrane test.

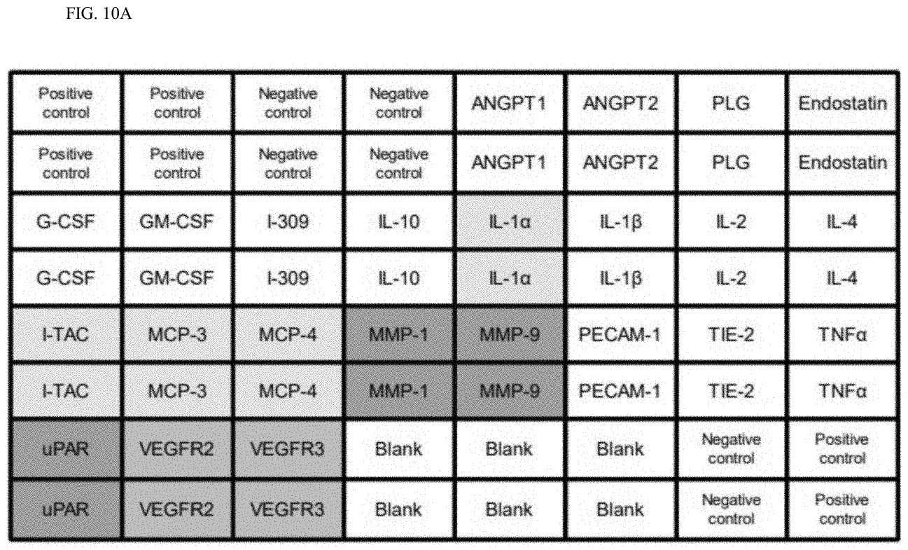

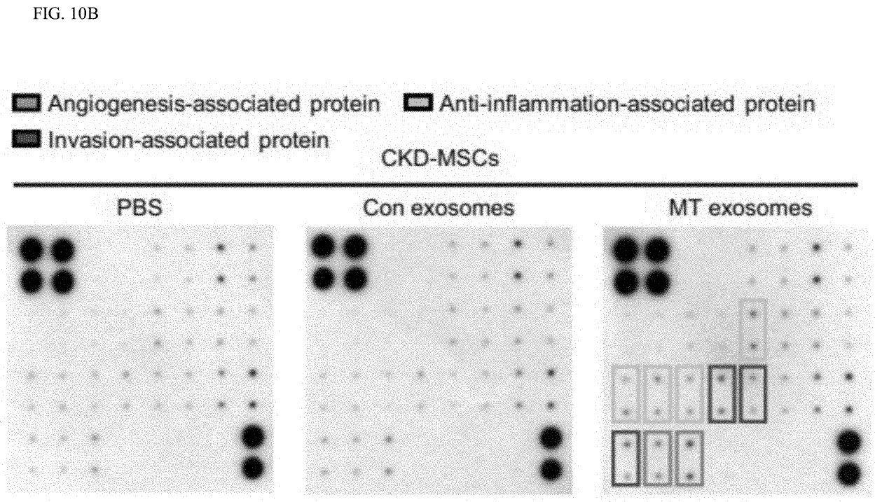

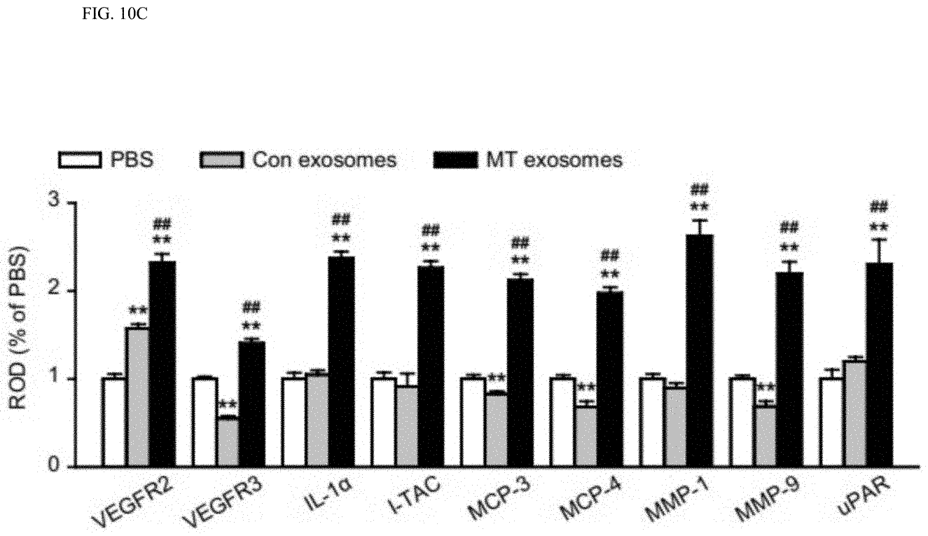

As a result, it was confirmed that the exosomes extracted from the mesenchymal stem cells derived from a healthy individual treated with melatonin increase the expression of angiogenesis-associated proteins, anti-inflammation-associated proteins, and invasion-associated proteins of the mesenchymal stem cells derived from a chronic kidney disease (CKD) patient (FIGS. 10A to 10C).

### Example 7. Confirmation of Therapeutic Effect of Mesenchymal Stem Cells Derived from a Chronic Kidney Disease (CKD) Patient on Autologous Cells Using Melatonin Exosomes in Murine Model with Chronic Kidney and Vascular Disease Complications

The autologous mesenchymal stem cells derived from a chronic kidney disease (CKD) patient pretreated with the pharmaceutical composition according to the present invention was transplanted into a murine hindlimb ischemia model, and on the 3^{rd} day, the apoptosis and cell proliferation in the transplanted site were respectively analyzed by TUNEL staining and proliferating cell nuclear antigen (PCNA) staining by confocal microscopy, and the blood flow ratio in the hindlimb region was confirmed through laser doppler for 28 days, and the degree of leg recovery in the hindlimb region into which stem cells were transplanted on the 28th day was evaluated. Additionally, to confirm the recovery of capillary blood vessels in the hindlimb ischemia region into which stem cells were transplanted on the 28th day, CD31 antibody targeting capillary blood vessels and alpha-SMA antibody targeting veins were fluorescently stained and analyzed by confocal microscopy.

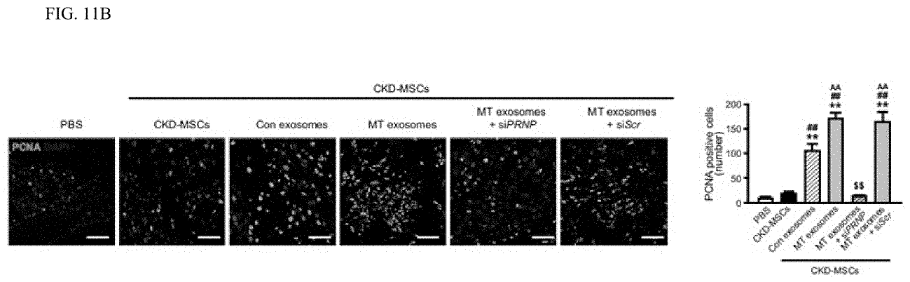

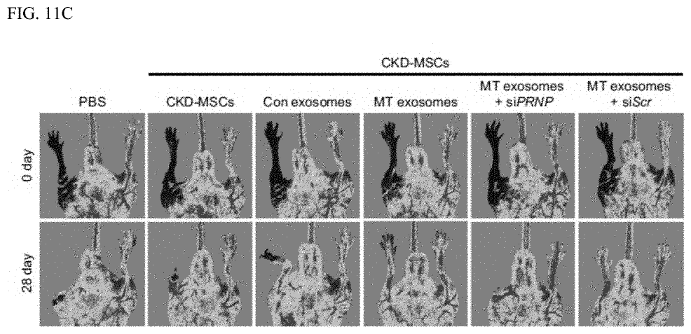

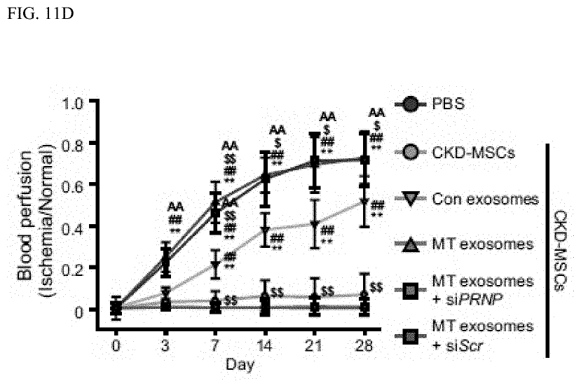

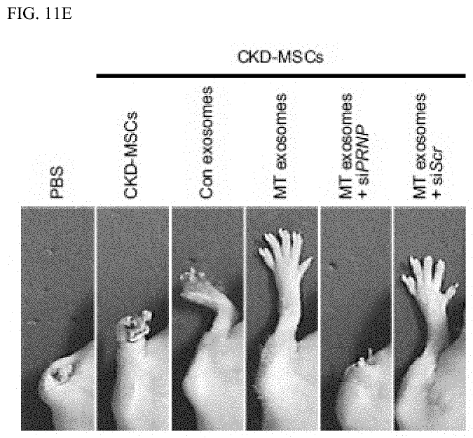

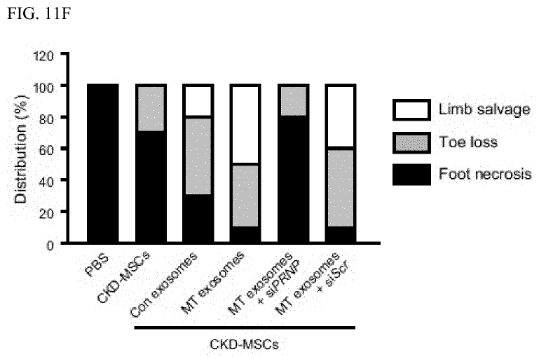

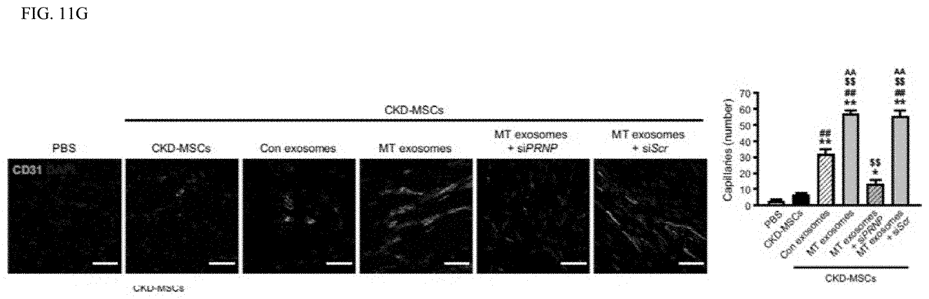

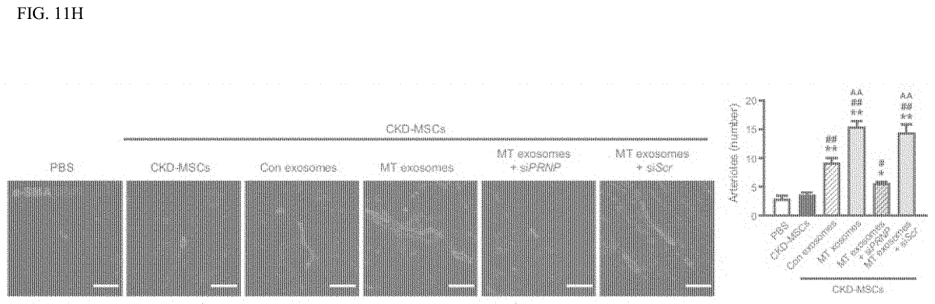

As a result, while in the melatonin exosome group, after the autologous mesenchymal stem cells derived from a chronic kidney disease (CKD) patient pretreated with the pharmaceutical composition according to the present invention were transplanted into a murine hindlimb ischemia model and the hindlimb ischemia region was biopsied on the 3^{rd} day, the apoptosis was reduced and cell proliferative potential was increased, in the PRNP siRNA melatonin exosome group, the apoptosis was increased but cell proliferative potential was decreased (FIGS. 11A and 11B), and on the 28^{th} day after transplantation, the ratio of blood flow in the hindlimb region was increased, whereas the amount of blood flow was decreased in the PRNP siRNA melatonin exosome group (FIGS. 11C to 11F). Additionally, with regard to degree of hindlimb recovery and angiogenesis on the 28^{th} day after transplantation, the melatonin exosome group showed an increase in hindlimb recovery and angiogenesis, whereas the PRNP siRNA melatonin exosome showed a decrease in hindlimb recovery and angiogenesis (FIGS. 11E to 11G).

The exosomes extracted from mesenchymal stem cells derived from a healthy individual co-cultured with melatonin or a culture solution thereof according to the present invention can increase the expression of the prion proteins within the autologous mesenchymal stem cells derived from a chronic kidney disease patient (see FIGS. 4A-6F), and can recover mitochondria functions (see FIGS. 7A-7F), cellular senescence (see FIGS. 8A-8D), and cell proliferative potential (see FIGS. 9A-9D), and additionally, can increase the expression of proteins associated with angiogenesis, anti-inflammation, and cell invasion (see FIGS. 10A and 10B). Accordingly, the exosomes extracted from mesenchymal stem cells derived from a healthy individual co-cultured with melatonin or a culture solution thereof according to the present invention are expected to be used as an aid for stem cell treatment of a patient with cardiovascular disease complications accompanying chronic kidney disease as well as a pharmaceutical composition for the effective treatment or prevention of chronic kidney disease.

What is claimed is:

1. A pharmaceutical composition for treatment or prevention of chronic kidney disease, comprising exosomes extracted from mesenchymal stem cells derived from a healthy individual co-cultured with melatonin or a culture solution thereof as an active ingredient.

2. The pharmaceutical composition of claim 1, wherein the mesenchymal stem cells are derived from umbilical cord, umbilical cord blood, bone marrow, fat, muscle, nerve, skin, amniotic membrane, or placenta.

3. The pharmaceutical composition of claim 1, wherein the pharmaceutical composition increases the expression of a prion protein.

4. The pharmaceutical composition of claim 1, wherein the pharmaceutical composition recovers mitochondrial functions, cellular senescence of stem cells, and cell proliferative potential of stem cells.

5. The pharmaceutical composition of claim 1, wherein the pharmaceutical composition increases the expression of proteins associated with angiogenesis of stem cells, anti-inflammation, and cell invasion.

6. The pharmaceutical composition of claim 1, wherein the pharmaceutical composition is used as an aid for the treatment of cardiovascular disease complications accompanying chronic kidney disease using autologous mesenchymal stem cells derived from a chronic kidney disease patient pretreated with the pharmaceutical composition.

7. A method for preparing a pharmaceutical composition for treatment or prevention of chronic kidney disease, wherein the method comprises:
(a) co-culturing mesenchymal stem cells derived from a healthy individual with melatonin;
(b) separating exosomes from the culture solution of Step (a); and
(c) preparing a composition comprising the exosomes separated in Step (b) as an active ingredient.

8. The method of claim 7, wherein the melatonin of Step (a) is contained in a medium at a concentration between 10⁻¹⁰ M or greater and 10⁻⁴ M or less.

9. The method of claim 7, wherein the separation of Step (b) is performed by centrifugation, ultracentrifugation, filtration by a filter, gel filtration chromatography, free-flow electrophoresis, capillary electrophoresis, separation using a polymer, and a combination thereof.

10. A method for promoting the proliferation of mesenchymal stem cells derived from a chronic kidney disease patient or increasing the survival rate of the patient, wherein the method comprises:
(a) co-culturing mesenchymal stem cells derived from a healthy individual with melatonin;
(b) separating exosomes from the culture solution of Step (a); and
(c) treating the exosomes separated in Step (b) to autologous stem cells derived from a chronic kidney disease patient of a mammal excluding humans and increasing the expression of a prion protein.

11. The method of claim 10, wherein the mesenchymal stem cells are derived from umbilical cord, umbilical cord blood, bone marrow, fat, muscle, nerve, skin, amniotic membrane, or placenta.

12. The method of claim 10, wherein the exosomes separated from the mesenchymal stem cells derived from a healthy individual recovers mitochondrial functions, cellular senescence, and cell proliferative potential.

13. The method of claim 10, wherein the exosomes separated from the mesenchymal stem cells derived from a healthy individual increases the expression of proteins associated with angiogenesis of stem cells, anti-inflammation, and cell invasion.
